(19) United States
(12) Patent Application Publication
(10) Pub. No.: US 2022/0192532 A1
(43) Pub. Date: Jun. 23, 2022

(54) POSITIONING OF A SUBCUTATEOUS DEVICE AND METHOD

(71) Applicants: KING ABDULLAH UNIVERSITY OF SCIENCE AND TECHNOLOGY, Thuwal (SA); STELLENBOSCH UNIVERSITY, Stellenbosch (ZA)

(72) Inventors: Jürgen KOSEL, Thuwal (SA); Liam SWANEPOEL, Thuwal (SA); Pieter FOURIE, Stellenbosch (ZA)

(21) Appl. No.: 17/600,414

(22) PCT Filed: Apr. 1, 2020
(86) PCT No.: PCT/IB2020/053108

§ 371 Date: Sep. 30, 2021

Related U.S. Application Data
(60) Provisional application No. 62/904,753, filed on Sep. 24, 2019.
(60) Provisional application No. 62/827,588, filed on Apr. 1, 2019.

(51) Int. Cl. A61B 5/06

(52) U.S. Cl. CPC A61B 5/062

(57) ABSTRACT

A subcutaneous medical device system includes a subcutaneous medical device, a magnetic element arranged on a portion of the subcutaneous medical device, the magnetic element including at least two poles, and a magnetic detector arranged spaced apart from the magnetic element and outside of a patient. The magnetic detector includes a single magnetic sensor and a processor coupled to the single magnetic sensor. The processor is programmed to determine a position of the portion of the subcutaneous medical device within the patient based on a static magnetic flux measurement of the magnetic element by the single magnetic sensor without externally applying an external magnetic field to the magnetic element.

### CROSS-REFERENCE TO RELATED APPLICATIONS

This application claims priority to U.S. Provisional Patent Application No. 62/827,588, filed on Apr. 1, 2019, entitled “A MAGNETIC METHOD FOR SUBCUTANEOUS DEVICE LOCALIZATION,” and U.S. Provisional Patent Application No. 62/904,753, filed on Sep. 24, 2019, entitled “POSITIONING OF A SUBCUTANEOUS DEVICE AND METHOD,” the disclosures of which are incorporated herein by reference in their entirety.

### BACKGROUND

### Technical Field

Embodiments of the subject matter disclosed herein generally relate to systems and methods for determining a position of a portion of a subcutaneous medical device based on a static magnetic flux of a magnetic element without applying an external magnetic field to the magnetic element.

### Discussion of the Background

Diagnosis and treatment of various medical ailments can involve the use of a subcutaneous medical device, i.e., a device that is inserted under the skin of a patient. Subcutaneous medical devices include catheters, as well as implanted medical devices that remain in the patient after surgery. Because a subcutaneous medical device is inserted below a patient's skin, the position of at least a portion of the device within the patient needs to be tracked to ensure the device is located in the desired position within the patient and that it avoids being in other positions within the patient that can result in injury.

One way of identifying the position of a portion of a subcutaneous medical device involves diagnostic x-ray examination. This, however, can injure the patient due to the use of contrast agents and exposure to x-ray radiation. Further, x-ray radiation requires a large amount of power and thus is only available in medical facilities.

Non-radiative techniques have been used to track the position of the tip of a catheter based on magnetic flux. One such technique involves affixing a magnet to the catheter tip so that it can rotate independently of the catheter tip. A motor rotates the magnet relative to the catheter tip to produce a magnetic oscillating field. This technique requires the use of at least two magnetic sensors to detect the oscillating magnetic fields in order to determine a position of the catheter tip within a patient. It appears this technique uses an oscillating magnetic field in order to more easily identify the magnetic flux of the magnet from the existing magnetic noise because the oscillation frequency can be isolated for identifying the magnetic flux produced by the oscillating magnetic field. Requiring the magnet to rotate relative to the catheter introduces an additional point of failure in the system, as well increases the costs and manufacturing complexity of the system. Further, this technique requires at least two magnetic sensors, which increases the size and weight of the device, as well as introduces additional costs and complexity to the system.

Other techniques involve the use of a device that can externally generate magnetic flux in coils located on the tip of a catheter. One implementation involves a plurality of magnetic field transducers arranged outside of the patient, which in conjunction with a magnetic field transducer on the tip of the catheter, can be used to determine the position and orientation of the catheter. This technique requires precise alignment of the external magnetic field transducers, which results in a rather complicated system. Another implementation involves the use of magnetic resonance signals from a magnetic resonance imaging (MRI) machine to generate the magnetic flux in the coils located on the tip of the catheter. MRI machines are large, expensive, and often are uncomfortable for patients. Employing coils on the tip of the catheter increases the size and weight of the catheter, and these coils consume power, which requires running wiring along the catheter to the coils.

Thus, there is a need for a low-complexity and cost system for determining the position of a portion of a subcutaneous medical device.

### SUMMARY

According to an embodiment, there is a subcutaneous medical device system, which includes a subcutaneous medical device, a magnetic element arranged on a portion of the subcutaneous medical device, the magnetic element including at least two poles, and a magnetic detector arranged spaced apart from the magnetic element and outside of a patient. The magnetic detector includes a single magnetic sensor and a processor coupled to the single magnetic sensor. The processor is programmed to determine a position of the portion of the subcutaneous medical device within the patient based on a static magnetic flux measurement of the magnetic element by the single magnetic sensor without applying an external magnetic field to the magnetic element.

According to another embodiment, there is a method for determining a position of a portion of a subcutaneous medical device within a patient. The subcutaneous medical device is inserted into the patient. A portion of the subcutaneous medical device includes a magnetic element, the magnetic element including at least two poles. A magnetic detector, which includes a single magnetic sensor and is arranged outside of the patient, is moved along the patient until static magnetic flux of the magnetic element is detected. Using a processor coupled to the single magnetic sensor, the position of the portion of the subcutaneous medical device within the patient is determined based on a measurement of the detected static magnetic flux of the magnetic element by the single magnetic sensor without applying an external magnetic field to the magnetic element.

According to a further embodiment, there is a method for determining a three-dimensional position of a portion of a subcutaneous medical device within a patient. The subcutaneous medical device is inserted into the patient. The subcutaneous medical device includes a magnetic element, the magnetic element including at least two poles. A static magnetic flux measurement is obtained from each magnetic sensor of a magnetic sensor array. Using a processor coupled to the magnetic sensor array, the three-dimensional position, inclination angle, and orientation of the subcutaneous medical device within the patient is determined based on a single static magnetic flux measurement from only two magnetic sensors of the magnetic sensor array without applying an external magnetic field to the magnetic element.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 is a schematic diagram of a subcutaneous medical device system according to embodiments;

FIG. 2 is a flow diagram of a method for determining the position of a portion of a subcutaneous medical device according to embodiments;

FIG. 3 is a flow diagram of a method for determining the position of a portion of a magnetic element according to embodiments;

FIG. 4 is a schematic diagram of a magnetic element and a graph illustrating the magnetic flux across the length of the magnetic element according to embodiments;

FIG. 5A is a graph illustrating a change in magnetic flux over a distance according to embodiments;

FIG. 5B illustrates a vector for determining orientation of a portion of a subcutaneous medical device according to embodiments;

FIG. 5C is a schematic diagram of a magnetic element and a graph illustrating the magnetic flux across the length of an inclined magnetic element according to embodiments;

FIGS. 6A and 6B are flow diagrams of methods for calibrating magnetic sensor measurements according to embodiments;

FIG. 7 is a schematic diagram of a subcutaneous medical device according to embodiments;

FIG. 8 is a flow diagram of a method for identifying particular portions of a subcutaneous medical device according to embodiments;

FIG. 9 is a flow diagram of a method for determining whether or not a subcutaneous medical device has been correctly inserted into a patient according to embodiments;

FIG. 10 is a block diagram of a magnetic sensor array according to embodiments;

FIG. 11 is a flow diagram of a method for determining the position of a portion of a subcutaneous magnetic device using a magnetic sensor array according to embodiments; and

FIG. 12 is a flow diagram of a method for identifying particular portions of a subcutaneous medical device using a magnetic sensor array according to embodiments.

### DETAILED DESCRIPTION

The following description of the exemplary embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to the terminology and structure of a subcutaneous medical device system.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

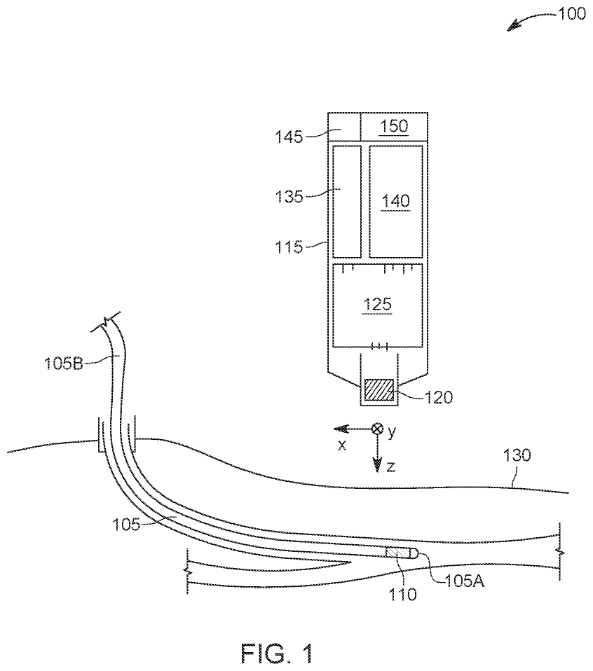

FIG. 1 is a schematic diagram of a subcutaneous medical device system according to embodiments. The subcutaneous medical device system 100 includes a subcutaneous medical device 105, a magnetic element 110 arranged on a portion of the subcutaneous medical device 105, and a magnetic detector 115 arranged spaced apart from the magnetic element 110 and outside of the patient 130. The magnetic detector 115 includes a single magnetic sensor 120 and a processor 125 coupled to the single magnetic sensor 120. The processor 120 is programmed to determine a position of the portion of the subcutaneous medical device 105 within the patient 130 based on a static magnetic flux measurement of the magnetic element 110 by the single magnetic sensor 120 without applying an external magnetic field to the magnetic element 110, i.e., without applying a direct and/or alternating current field to the magnetic element 110. The magnetic element 110 can include two poles (i.e., a dipole) or more than two poles (i.e., a multipole). The single magnetic sensor 120 can be, for example, any type of magnetic sensor that does not require generation of magnetic fields for the magnetic sensor 120 to detect static magnetic flux from the magnetic element 110. Further, the single magnetic sensor 120 can be a single-axis sensor (e.g., configured to detect magnetic flux along the z-axis, which is perpendicular to the patent 130) or can be a multi-axis sensor. The term patient should be understood as including any type of animal, including humans and other animals. In the illustrated embodiment, the system includes only a single magnetic element 110 arranged on the subcutaneous medical device 105.

In the illustrated embodiment the subcutaneous medical device 105 is a catheter having a proximate end 105A with a tip and an opposite distal end 105B, with the magnetic element 110 being located at the proximate end 105A. The magnetic element 110 is located on the proximate end 105A of the catheter because the position of the tip of the catheter is the primary concern when using a catheter. The catheter can be any type of catheter, such as, for example, an umbilical catheter, a urinary catheter, an endotracheal tube, subcostal drain, a vetriculo-peritoneal shunt, and peripherally inserted central catheter (PICC). It should be recognized that the magnetic element can be affixed to other types of subcutaneous medical devices, including arterial-venous shunts, artificial heart valves, artificial arteries (such as those placed in an aortic aneurism), and stents. The location at which the magnetic element is attached for these other types of subcutaneous medical devices can vary, depending upon the portion of the device for which the position is desired. Furthermore, some subcutaneous medical devices may be short enough that the magnetic element can be located anywhere along the subcutaneous medical device so that the position of a portion of the device can be determined, i.e., any location along the short subcutaneous medical device is sufficient to determine whether the subcutaneous medical device is properly located within the patient's body.

The magnetic element 110 can be any type of structure that can produce a static magnetic flux without the need to apply an external magnetic field to the magnetic element 110 (i.e., without applying a direct current or alternating current field using external coils or applying electricity directly to the magnetic element 110), including a purely metallic magnet, an elastic polymer, a polymer compound, a ceramic magnet, a structure (e.g., a flexible structure, such as an elastic polymer) coated or filled with magnetic particles, etc. One example of a polymer magnetic element 110 is one that comprises silicon and a magnetic micro-powder of NdFeB, which is advantageous because the material is capable of sustaining a large remanence magnetization. The use of a polymer magnetic element 110 is particularly advantageous when the magnetic element is located on the outer periphery of the subcutaneous medical device 105 because the magnetic element 110 is soft and will deflect when it impinges on soft tissue. Further, it is light and will not interfere with the subcutaneous medical device 105 dynamics (i.e., the movement of the subcutaneous medical device 105 inside of the patient 130). When the magnetic element 110 is located on the outer periphery of the subcutaneous medical device 105, the wall thickness of the magnetic element 110 (i.e., the thickness in a direction perpendicular to the length of the subcutaneous medical device 105) should be thin enough so that it does not interfere with the insertion and functionality of the subcutaneous medical device 105.

In the illustrated embodiment, the magnetic element 110 is located on the outer periphery of the subcutaneous medical device 105, which allows the system to be retrofitted on an existing subcutaneous medical device that was not originally designed to operate in this system. Of course, the magnetic element 110 can be arranged on the outer periphery of the subcutaneous medical device 105 as part of the manufacturing process. However, the magnetic element 110 can also be arranged inside of the subcutaneous medical device 105 so as to not interrupt the outer periphery of the device, such as, for example, being injection molded within the catheter. In either embodiment, the magnetic element 110 is attached to the subcutaneous medical device 105 so that the magnetic element 110 and the subcutaneous medical device 105 fixedly rotate together.

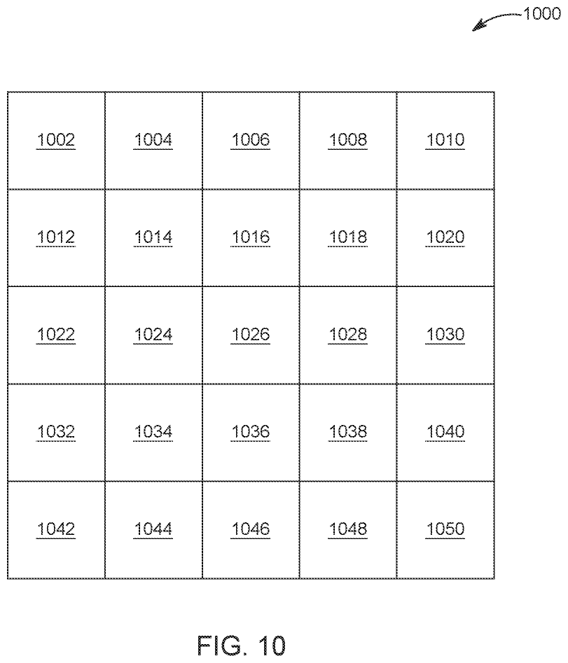

As illustrated in FIG. 1, the magnetic detector 115 can also include a battery 135 (if the detector is operated wirelessly), connective and operational circuitry 140 (which includes, for example, a circuitry for signal conditioning, filtering, amplification/preamplification, and power regulation), a visual signal strength indicator 145, and an audible signal strength indicator 150. The visual signal strength indicator 145 can be any type of visualization to indicate the relative magnitude of the detected static magnetic flux, such as a bar that increases or decreases in size depending upon the magnitude of the detected static magnetic flux. It may also be used to indicate directional information related to the catheter, such as an arrow on a display that points in the direction of the catheter orientation. It may also be used to indicate positional information related to the catheter in the case of a sensor array (described in more detail below in connection with FIG. 10), such as an array of lights which is on at the location of the catheter. Similarly, the audible signal strength indicator 150 can be a speaker that provides any type of audible indication of the magnitude of the detected static magnetic flux, such as a sound or tone that increases or decreases in frequency, pitch, and/or volume depending upon the magnitude of the detected static magnetic flux. The magnetic detector 115 need not include both a visual and audible signal strength indicator but instead can include only one or the other.

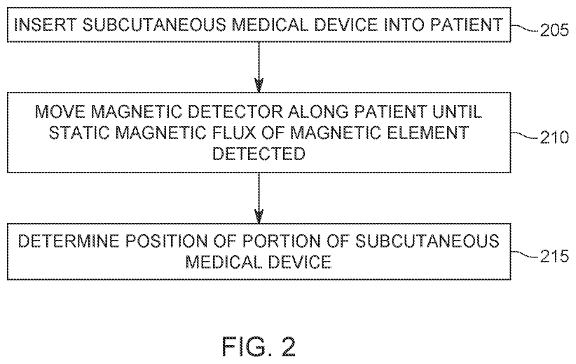

A method for using the system illustrated in FIG. 1 will now be described in connection with the flowchart of FIG. 2. Initially, the subcutaneous medical device 105 is inserted into the patient 130 (step 205). A portion of the subcutaneous medical device 105 includes a magnetic element 110 having at least two poles. A magnetic detector 115, which includes a single magnetic sensor 120 and is arranged outside of the patient 130, is moved along the patient 130 until a static magnetic flux of the magnetic element 110 is detected (step 210). Using a processor 125 coupled to the single magnetic sensor 120, the position of a portion of the subcutaneous medical device 105 within the patient 130 is determined based on a measurement of the detected static magnetic flux of the magnetic element 110 by the single magnetic sensor without applying an external magnetic field, i.e., without applying a direct and/or alternating current field to the magnetic element 110 (step 215). The determined position can be a two-dimensional position, i.e., an x-y position, or a three-dimensional position, i.e., an x-y-z position.

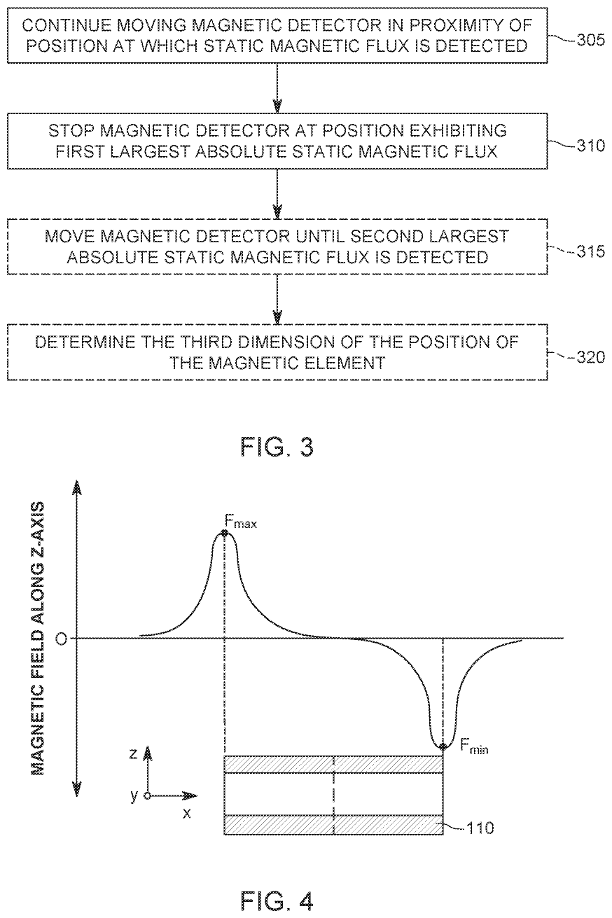

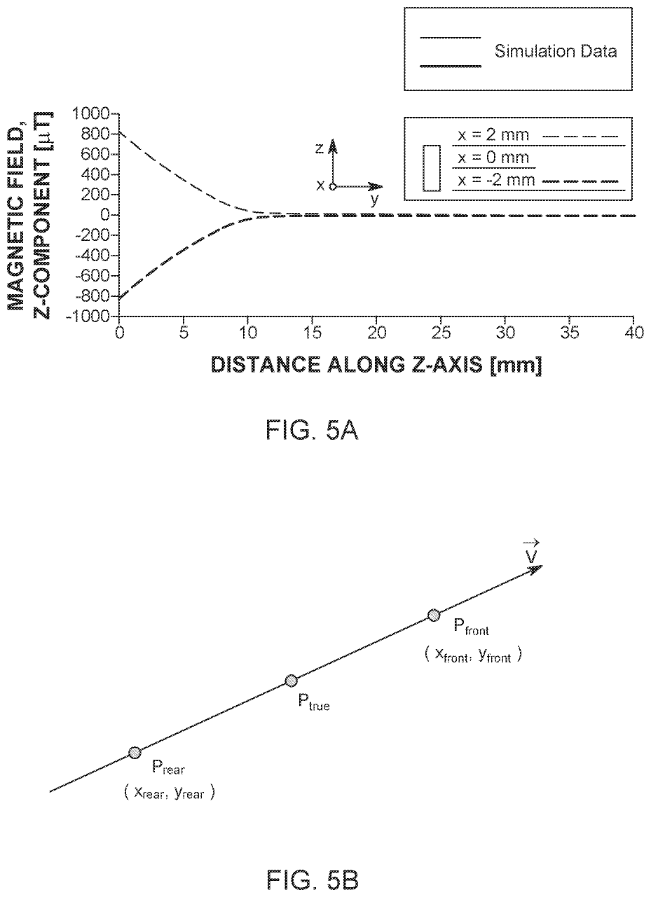

A method for determining the two- or three-dimensional position of a portion of the magnetic element in step 215 will now be discussed in connection with FIGS. 3-5A. Once the static magnetic flux of the magnetic element 110 is detected, the magnetic detector 115 is moved in proximity of this position (step 305) and it is stopped at the position exhibiting the first largest absolute static magnetic flux (step 310). Specifically, referring to FIG. 4, the magnetic element 110 has two poles (the dashed line schematically illustrates the division between these poles), one of which exhibits a largest static magnetic flux (Fₘₐₓ) and another of which exhibits the smallest static magnetic flux (Fₘᵢₙ). Thus, if in step 210 a magnetic flux measurement is obtained that is not the largest or smallest magnetic flux, then the magnetic detector 115 is moved in proximity of this location until the largest (Fₘₐₓ) or smallest (Fₘᵢₙ) is detected, i.e., either of these could be the first largest absolute static magnetic flux.

In some cases, identifying the position exhibiting the largest absolute static magnetic flux may be sufficient for identifying the position of a portion of the subcutaneous medical device 105, such as when the position of the tip of the catheter is desired, and thus the method can stop at step 210. In other cases, it may be desirable to identify the position of the entire length of the magnetic element 110 or the midpoint of the magnetic element 110. In these cases, the magnetic detector 115 is then moved until the second largest absolute static magnetic flux is detected (step 315). Thus, if the first largest absolute static magnetic flux corresponds to the north pole of the magnetic element 110, then the second largest absolute static magnetic flux corresponds to the south pole of the magnetic element 110, and vice-versa. If the position of the middle of the magnetic element is desired, the midpoint will be the point between the first and second largest absolute magnetic fluxes, which should exhibit almost no static magnetic flux. As used in connection with this method, as well as in the description that follows, the terms “first largest” and “second largest” with respect to absolute static magnetic flux are used to differentiate between the two largest absolute magnetic flux fields of the magnetic element 110 and is not intended to indicate that the value of one of these absolute static magnetic flux values is larger than the other.

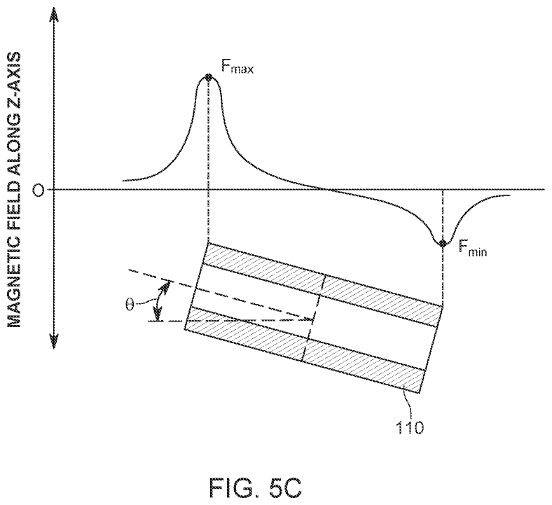

Returning to FIG. 3, the position of the first largest (or the first and second largest) absolute static magnetic flux identifies the x-y position of the portion of the subcutaneous medical device 105 on which the magnetic element 110 is mounted. If the x-y position is all that is required, then the method can end after step 310 or 315 (if step 315 is performed). If, however, a three-dimensional position is required, then the third dimension of the position of a portion of the magnetic element 110 is determined (step 320). The third dimension of the position is determined based on the magnitude of the measured static magnetic flux at the position of the largest absolute static magnetic flux measurement, i.e., the determination of the z-position, as well as the x- and y-positions, can be performed using only a single static magnetic flux measurement of the single magnetic sensor 120. The largest absolute static magnetic flux can be the largest or smallest static magnetic flux measurement. Specifically, referring now to the graph of FIG. 5A, the absolute magnitude of the static magnetic flux decreases as the distance between the single magnetic sensor 120 and the magnetic element 110 increases—the static magnetic flux drops as a function of the distance from the magnetic element 110, as illustrated in FIG. 5A. Thus, knowing the absolute magnitude of the first and/or second largest absolute static magnetic flux of one or more poles of the magnetic element 110 when the single magnetic sensor 120 is directly adjacent to the magnetic element 110 allows one to determine the z-coordinate based on the amount of attenuation in the magnitude of the static magnetic flux due to the increased distance between the single magnetic sensor 120 and the magnetic element 110. The determination of the absolute magnitude of the static magnetic flux when the single magnetic sensor 120 is directly adjacent to the magnetic element 110 can be performed prior to inserting the subcutaneous medical device 105 into the patient, i.e., prior to step 205. For example, the absolute magnitude can be based upon specifications provided by a manufacturer or distributor or the magnetic element 110 or can be based upon a single measurement prior to insertion of the subcutaneous medical device 105 into the patient.

In addition to using a single magnetic sensor 120 to determine the two- or three-dimensional position of a portion of a subcutaneous medical device 105, the orientation and inclination angle of the subcutaneous medical device 105 can be determined using the single magnetic sensor 120, which will now be described in connection with FIGS. 5B and 5C, respectively. Turning first to FIG. 5B, the subdermal position of the center of the magnetic element 110 is located at:

$${P}_{true} = ( \frac{{( {P}_{rear} - {P}_{front} )}_{x}}{2} , \frac{{( {P}_{rear} - {P}_{front} )}_{y}}{2} ) \qquad ( 1 )$$

where Pₜᵣᵤₑ is the x-y position of the middle of the magnetic element 110 between the two pole ends, front is P_{front} the x-y position of the front of the magnetic element 110, and rear is Pᵣₑₐᵣ the rear of the magnetic element 110.

The magnetic element 110, and thus the portion of the subcutaneous medical device 105 on which the magnetic element 110 is arranged, is aligned along the vector:

V_{rear-front}=[(P_{front}−Pᵣₑₐᵣ)_{x′}(P_{front}−Pᵣₑₐᵣ)_{y}] (2)

Accordingly, the vector along the position of the two largest absolute static magnetic flux measurements (i.e., Fₘₐₓ and Fₘᵢₙ) determines the orientation of the portion of the subcutaneous medical device 105 on which the magnetic element 110 is arranged. If the position of one particular end of the magnetic element 110 is desired, such as when it is desired to know the exact position of the tip of a catheter, this may be determined by summing half the length of the magnetic element 110 along the directional vector V_{rear-front}. Specifically, the maximum static magnetic flux of the magnetic element 110 is slightly beyond the length of the magnetic element itself and the distance between the position where the maximum static magnetic flux is detected and the actual end of the magnetic element increases as the magnetic sensor 120 is moved further away from the magnetic element 110. Thus, while identifying the position at which the maximum static magnetic flux is detected may be sufficient for some uses, in other uses this method can be used for determining a more precise position of the actual end of the magnetic element 110.

Turning now to FIG. 5C, the inclination angle of the magnetic element 110 can be determined based on the relative magnitudes of the two largest absolute static magnetic flux measurements (Fₘₐₓ and Fₘᵢₙ). As discussed above, the depth of the magnetic element 110 can be determined based on a comparison of the measured largest absolute static magnetic flux measurement Fₘₐₓ and Fₘᵢₙ with a reference measurement made with the single magnetic sensor 120 directly adjacent to one of the poles of the magnetic element 110. This can be extended for use in determining the inclination angle. Specifically, as illustrated in FIG. 5C, when the magnetic element 110 is inclined the measured absolute value of the static magnetic flux of the two poles, when measured by the single magnetic sensor 120 at the same distance from the patient in the z-direction, will have different values. In the illustrated embodiment the absolute static magnetic flux value of Fₘₐₓ is larger than that of Fₘᵢₙ because the pole of Fₘₐₓ is closer to the magnetic detector 115 than pole of Fₘᵢₙ. Accordingly, the depth of each side of the magnetic element 110 can be determined by comparing the measured largest absolute static magnetic flux measurement Fₘₐₓ and Fₘᵢₙ with a reference measurement made with the single magnetic sensor 120 directly adjacent to one of the poles of the magnetic element 110. The angle of inclination can then be calculated based on a vector between the determined depths of the two ends of the magnetic element 110. The determination of the orientation and/or inclination of the magnetic element 110, which also provides the orientation and/or inclination of the portion of the subcutaneous medical device 105 on which the magnetic element 110 is arranged, can be performed in connection with the determination of the two- or three-dimensional position of a portion of the subcutaneous medical device 105 in step 215. Further, the determination of the two- or three-dimensional position, orientation, and inclination angle can be determined using a single, single-axis magnetic sensor 120.

The method described above in connection with FIG. 5C involved a calibration step to obtain the reference measurement that is subsequently used to determine the inclination angle and depth of the magnetic element 110. Another method can be employed for determining the angle of inclination that avoids the need to obtain a reference measurement by comparing the two peak magnitudes Fₘₐₓ and Fₘᵢₙ. Correlating the measured field intensities to the angles of inclination first involves calculating the difference and the average of the peak values as follows:

$$\begin{matrix}Δ = ( {F}_{max} - \vert {F}_{min} \vert ) & ( 3 ) \\ \overline{A} = \frac{( {F}_{max} + \vert {F}_{min} \vert )}{2} & ( 4 )\end{matrix}$$

Next, the angle of inclination can be calculated as follows: n=Δ/Ā. With an increase in inclination angle, the ratio of the peak difference A increases relative to the average of the peak values Ā. This allows the approximation of the angle of inclination, regardless of the depth of the subcutaneous medical device. Subsequently, the depth of the tip of the subcutaneous medical device can be determined using supervised machine learning, such as a depth classification function. The classification function is specific to each inclination angle and correlates the average magnetic field measurement Ā to a depth of the subcutaneous medical device. Classification functions are established for each tip size and composition of the subcutaneous medical device. Specifically, each classification function correlates the average magnetic field measurement Ā to a placement depth estimation. The classification function is built in advance based on the tip size and composition of the particular subcutaneous medical device that is being employed. If the system is designed to employ different types of subcutaneous medical devices, then multiple classification functions can be built, one for each type of subcutaneous medical device that can be employed in the system. Alternatively, a regression function could be used in a similar manner.

Thus, a method using the technique above can involve moving a magnetometer relative to a patient in order to identified the first and second largest absolute static magnetic flux measurements (i.e., Fₘₐₓ and Fₘᵢₙ). Next, the difference between the first and second largest absolute static magnetic flux measurements (A) and the average of the first and second largest absolute static magnetic flux measurements (Ā) are determined. The angle of inclination is then determined based on a ratio of the difference between the first and second largest absolute static magnetic flux measurements (Δ) to the average of the first and second largest absolute static magnetic flux measurements (Ā). The depth of the subcutaneous medical device can then be determined using a depth classification function.

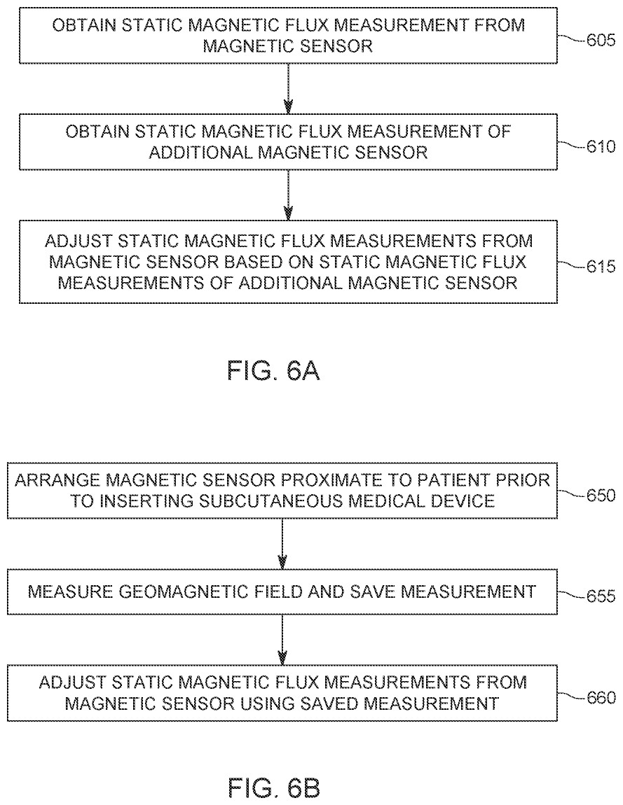

In order for the magnetic element 110 to be compatible with a subcutaneous medical device, the magnetic element 110 must be sized appropriately. For example, when the subcutaneous medical device 105 is an intravenous catheter and the magnetic element 110 is arranged on the outer periphery of the catheter, the magnetic element 110 can have, for example, 1 mm diameter and can be 5 mm in length. A magnetic element of this size typically exhibits a static magnetic flux having an absolute magnitude smaller than the magnitude of the magnetic flux of the earth's geomagnetic field. Thus, in order to more accurately detect the static magnetic flux of the magnetic element, the effects of the geomagnetic field should be accounted for, examples methods of this are illustrated in FIGS. 6A and 6B. The method of FIG. 6A involves using two magnetic sensors during the position determination, whereas the method of FIG. 6B involves using a single magnetic sensor to perform measurements before and during the insertion of the subcutaneous medical device.

Turning first to FIG. 6A, the method involves one magnetic sensor that is used to measure the magnetic element 110 and another magnetic sensor, which is sufficiently spaced apart from the magnetic element 110 so as to not be affected by the static magnetic flux of the magnetic element 110. Thus, static magnetic flux measurements are obtained from the magnetic sensor used for identifying the position of a portion of the subcutaneous medical device 105 (step 605). At the same time, prior to, or subsequent to the measurement of the static magnetic field of the magnetic element 110, a static magnetic flux measurement of the geomagnetic field is obtained by another magnetic sensor (step 610). The static magnetic flux measurements from the magnetic sensor used for identifying the position of a portion of the subcutaneous medical device 105 are subtracted from the static magnetic flux measurements obtained from the other magnetic sensor so as to cancel the magnetic flux induced into the magnetic flux measurements of the magnetic element that are induced by the geomagnetic field (step 615). Thus, the method of FIG. 6A will be performed concurrently with the method of FIG. 1.

Turning now to FIG. 6B, the method involves using a single magnetic sensor to measure both the geomagnetic field and the static magnetic flux of the magnetic element. Specifically, the magnetic sensor 120 is arranged proximate to the patient prior to inserting the subcutaneous medical device 105 (step 650) and the geomagnetic field is measured and saved (step 655). After the subcutaneous medical device 105 is inserted into the patient (i.e., after step 105 in FIG. 1), the static magnetic flux measurements by the magnetic sensor 105 are adjusted by subtracting the magnitude of the measured magnetic flux of the geomagnetic field so that the magnetic flux of the geomagnetic fields is removed from the measurements of the magnetic element 110 (step 660). The methods of FIGS. 6A and 6B can be used in conjunction with the method of FIG. 2.

As will be appreciated from the discussion above, the disclosed subcutaneous medical device system and method provides a relatively simple way to determine the position of a portion of a subcutaneous medical device because it only requires a single magnetic element on the subcutaneous medical device and a single magnetic sensor. This significantly expands the possible uses of the disclosed systems and methods. Specifically, although many subcutaneous medical devices are inserted into patients while in a medical facility, some devices must be inserted when the patient is not located in a medical facility. For example, patients are often intubated with an endotracheal tube when medical responders first arrive to treat the patient. If the endotracheal tube is inserted too far into the patient it could damage the lungs or ventilate only one lung, and if it is not inserted far enough into the patient it can fail to operate as intended. Thus, by relying on only a single magnetic sensor and a single magnetic element that does not require an externally applied magnetic field, the disclosed system can be made compact and battery powered and can be employed anywhere, such as at the scene of an accident, in a medical transport (e.g., an ambulance, medical helicopter, medical plane, etc.). In contrast, prior techniques required complicated equipment, which are large and not suitable for use outside of a medical facility and require a large amount of power beyond what can be supplied by a reasonably sized battery.

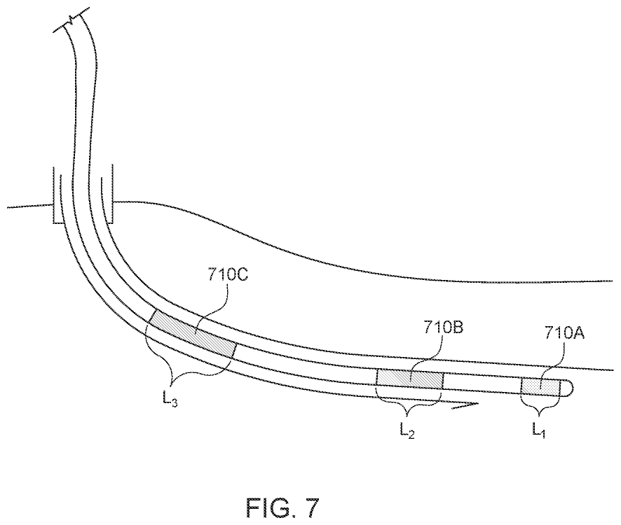

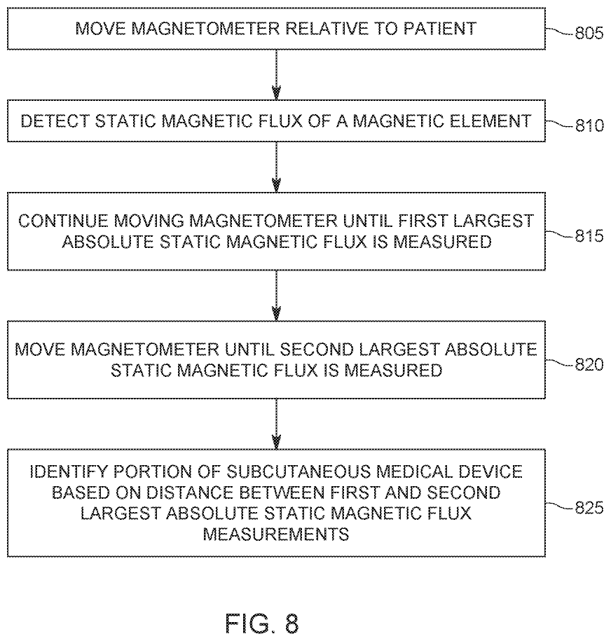

The discussion above involves identifying the position of a portion of a subcutaneous medical device using a single magnetic element. This can be useful with small subcutaneous medical devices or those were only a portion needs to be monitored (e.g., the tip of a catheter). In other situations, it may be desirable to determine the position of a number of different portions of a subcutaneous medical device or a different portion of the subcutaneous medical device. For example, ventriculoperitoneal shunts are placed inside of some children shortly after birth. However, as the child grows the catheter may become dislodged from the initial placement site, which causes the catheter not to function as intended. By monitoring the specific catheter length section opposite of a specific anatomical landmark, this technique provides an accurate and cost-effective way to monitor catheter movement. Thus, a magnetic element placed on the tip of the catheter can be used for positioning for inserting the catheter into the correct position within the patient and then another magnetic element arranged on a different portion of the catheter can be used for in-situ monitoring after the catheter has been successfully placed within the patient. A system and method for doing so will now be described in connection with FIGS. 7 and 8.

The position of number of portions of a subcutaneous medical device can be determined by using “coded” magnetic elements. Specifically, referring to FIG. 7, a number of different magnetic elements 710A-7100 can be placed along different portions of the subcutaneous medical device. Each of these magnetic elements 710A-7100 have a different length, which in the illustrated embodiment is lengths L1-L3, respectively. These different lengths are used to identify the particular portion. As discussed above in connection with FIG. 4, a magnetic element exhibits its maximum positive magnetic flux at the north end of the element and a maximum negative magnetic flux at the south end of the element. Thus, by identifying these maximums and the length between them, the particular magnetic element can be identified based on its length, i.e., the length between the maximum positive and maximum negative magnetic flux.

A method for identifying a particular portion of a subcutaneous medical device using a single magnetic sensor will now be described in connection with FIG. 8. Initially, the magnetic sensor 120 (not illustrated) is moved relative to the patient (step 805) until the static magnetic flux of an element is identified (step 810). For purposes of discussion, it is assumed that the static magnetic flux that is identified is not from one of the poles of the magnetic element 110. The magnetic sensor 120 is then moved until the first largest absolute static magnetic flux (Fₘₐₓ or Fₘᵢₙ) is measured by the magnetic sensor 120 (step 815). The magnetic sensor 120 is then moved until the second largest absolute static magnetic flux (Fₘₐₓ or Fₘᵢₙ) is measured by the magnetic sensor 120 (step 820). The portion of the subcutaneous medical device 105 can then be identified based on the distance between the first and second largest absolute static magnetic flux measurements (step 825). This involves comparing the distance with the length of the different magnetic elements 710A-7100 on the subcutaneous medical device 105 and the magnetic element 710A-7100 having a length corresponding with the distance identifies the portion of the subcutaneous medical device 105 detected by the magnetic sensor. The method of FIG. 8 can be used in conjunction with the methods of FIGS. 2, 3, 6A, and 6B. For example, the method of FIG. 8 can be performed before, during, or after the method of FIG. 2. Thus, the method of FIG. 8 can involve measuring the static magnetic flux of the magnetic element over a period of time while the magnetic sensor (or array of magnetic sensors—see discussion in connection with FIG. 10 below) and the magnetic element are in a fixed position relative to each other and determining whether or not the subcutaneous medical device is inserted into an artery in the patient based on changes in magnetic flux of the magnetic element over the period of time.

In addition to, or as an alternative to, using the disclosed subcutaneous medical device system to determine the position of a portion of a subcutaneous medical device, the system can also be used to determine whether the subcutaneous medical device has been correctly inserted into the patient. Specifically, due to the alternating high and low pressure within an artery it is believed that when a subcutaneous medical device, having a magnetic element, is inserted into an artery instead of a vein the static magnetic flux will change over time according to the amount of pulsatile blood passing by the magnetic element.

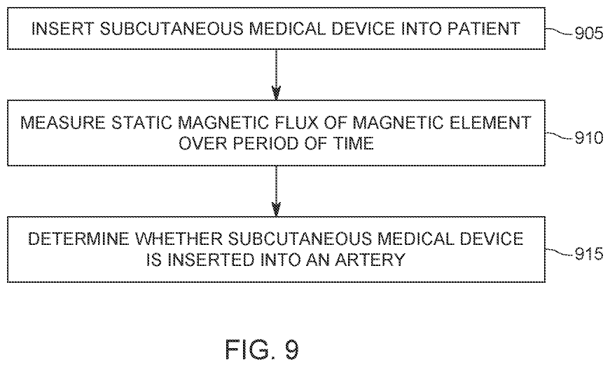

A method for determining whether a subcutaneous medical device is correctly inserted in a patient using this recognition will now be described in connection with FIG. 9. Initially, a subcutaneous medical device 105 is inserted into the patient 130 (step 905). The subcutaneous medical device 105 includes a magnetic element 110. A magnetic sensor 120 is used to measure the static magnetic flux of the magnetic element 110 over a period of time while the magnetic sensor 120 and magnetic element 110 are in a fixed position relative to each other (step 910). A processor 125 coupled to the magnetic sensor 120 determines whether or not the subcutaneous medical device 105 is inserted into an artery in the patient based on changes in the static magnetic flux of the magnetic element 110 over the period of time (step 915). Specifically, due to alternating high and low pressure within an artery, the static magnetic flux should vary over time if the subcutaneous medical device 105 is inserted in an artery, whereas this change in pressure does not occur in a vein and thus the static magnetic flux measurements in a vein should not vary over time if the subcutaneous medical device 105 is inserted into a vein. The method of FIG. 9 can be used in conjunction with the methods disclosed above. For example, the method of FIG. 9 can be performed first and, after confirming that the subcutaneous medical device 105 is inserted into a vein, the method of FIG. 2 can be performed, starting at step 210.

The embodiments discussed above involve determining a two- or three-dimensional position using only a single magnetic sensor, which is particularly advantageous because it provides a low-cost and relatively simple system for determining the two- or three-dimensional position of a portion of a subcutaneous medical device. The ability to determine a three-dimensional position using only a single magnetic sensor is a significant improvement over prior techniques that typically required assigning a sensor to each positional axis so that three different sensors are required to determine a three-dimensional position. The disclosed techniques can also be used with multiple magnetic sensors. For example, referring to FIG. 10, an array of magnetic sensors 1002-1050 can be employed. The array of magnetic sensors can either be arranged in a fixed position relative to the patient or can be moved relative to the patient. Although the array of magnetic sensors 1002-1050 are illustrated as being in a square array, any other shaped array can be employed, such as, for example, a circular array. The array of magnetic sensors 1002-1050 can, for example, be arranged on a transparent substrate that allows for visually identifying the position of a portion of the subcutaneous medical device 105 from the non-sensing side of the magnetic sensors 1002-1050. In this case, the magnetic elements of the magnetic detector 115 need not be integrated in a common housing with the other components, as is the case when a single magnetic detector is used as discussed above. The magnetic sensors of the array can be, for example, magnetometers. Further, the magnetic sensors of the array can be single-axis sensors (e.g., configured to detect magnetic flux along the z-axis, which is perpendicular to the patent 130) or can be multi-axis sensors.

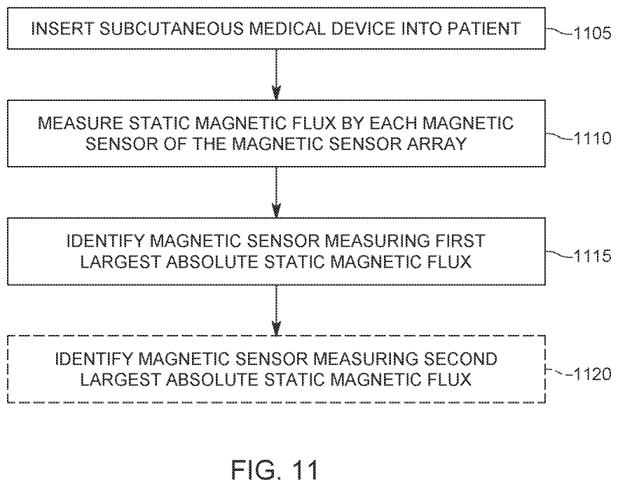

A method for using the system illustrated in FIG. 1 with an array of magnetic sensors 1002-1050 will now be described in connection with the flowchart of FIG. 11. After the subcutaneous medical device 105 is inserted into the patient 130, each magnetic sensor 1002-1050 of the magnetic sensor array 1000 obtains a static magnetic flux measurement (step 1110). The sensor with the first largest absolute static magnetic flux measurement is identified and can be used as the x-y position of the portion of the subcutaneous medical device 105 on which the magnetic element 110 is arranged (step 1115). The z-position can be determined using the techniques discussed above in connection with FIG. 5A. Thus, the three-dimensional position of a portion of the subcutaneous medical device 105 can be determined using the single static magnetic flux measurement of one of the magnetic sensors 1005-1050 of the array. If desired, this embodiment can be employed to determine a two-dimensional position instead of a three-dimensional position.

An additional optional step can be to identify the second largest absolute static magnetic flux measurement (step 1120). This additional step can be performed if the position of the entirety (or the midpoint between poles) of magnetic element 110 is desired. Furthermore, this additional optional step allows the determination of the orientation and/or the inclination of the magnetic element 110 using the techniques discussed above in connection with FIGS. 5B and 5C. Accordingly, using an array of magnetic sensors 1005-1050 the two- or three-dimensional position, orientation, and inclination angle of the subcutaneous medical device can be determined using only a single static magnetic flux measurement from only two magnetic sensors 1005-1050 of the array.

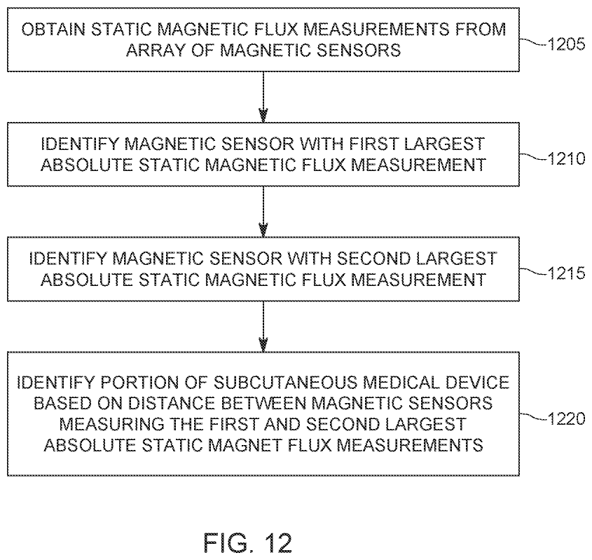

The array of magnetic sensors 1005-1050 can also be used to identifying a particular portion of a subcutaneous medical device 105 in a similar manner to that discussed above in connection with FIGS. 7 and 8. Specifically, referring now to FIGS. 7 and 12, static magnetic flux measurements are obtained from each magnetic sensor in the array of magnetic sensors (step 1205). Using these measurements, the magnetic sensor having the first largest absolute static magnetic flux measurement (step 1210) and the magnetic sensor having the second largest absolute magnetic flux measurement (step 1215) are identified. The portion of the subcutaneous medical device 105 can be identified based on the distance between the magnetic sensors measuring the first and second largest static magnetic fluxes (step 965). Similar to the method of FIG. 8, this involves comparing the distance with the length of the different magnetic elements 710A-710C on the subcutaneous medical device 105 and the magnetic element 710A-710C having a length corresponding with the distance identifies the portion of the subcutaneous medical device 105 detected by the magnetic sensors.

Because the method of FIG. 12 involves the use of an array of magnetic sensors that, in some embodiments, cover a substantial portion of the patient's body and there are more than one magnetic elements arranged on the subcutaneous medical device, the method of FIG. 12 may result in measurements from a number of magnetic sensors having a first largest absolute static magnetic flux and a measurements from a corresponding number of magnetic sensors having a second largest absolute static magnetic flux. In this case, the locations of the different magnetic sensors are used to determine which magnetic sensors are detecting which magnetic elements. For example, referring to FIG. 10, assuming that magnetic sensors 1014 and 1016 respectively measure the largest and smallest static magnetic flux compared surrounding magnetic sensors (i.e., 1002-1008, 1012, 1018, and 1022-1028) it can be determined that magnetic sensors 1014 and 1016 are measuring the same magnetic element. Similarly, if magnetic sensors 1036 and 1038 respectively measure the largest and smallest static magnetic flux compared surrounding magnetic sensors (i.e., 1024, 1026, 1028, 1030, 1034, 1040, 1044, 1046, 1048, and 1050) it can be determined that magnetic sensors 1014 and 1016 are measuring the same magnetic element. The method of FIG. 12 can be used in conjunction with the methods of FIGS. 2, 3, 6A, and 6B. For example, the method of FIG. 12 can be performed before, during, or after the method of FIG. 2.

It should be recognized that the methods of FIGS. 6A and 6B for determining the earth's geomagnetic field can be used with the magnetic sensor array 1000. In the method of FIG. 6A, the magnetic sensor can be any one of the magnetic sensors of the array and the additional magnetic sensor can be a separate magnetic sensor. In the method of FIG. 6B, the magnetic sensor used to measure the geomagnetic field prior to inserting the subcutaneous medical device into the patient can be the same or different from the magnetic sensor of the magnetic sensor array having the static magnetic flux measurements adjusted by the geomagnetic field measurement.

Although the techniques are disclosed in connection with a subcutaneous medical device, these techniques can be used in other applications with other types of devices.

The disclosed embodiments provide systems and methods for determining the position of a portion of a subcutaneous medical device. It should be understood that this description is not intended to limit the invention. On the contrary, the exemplary embodiments are intended to cover alternatives, modifications and equivalents, which are included in the spirit and scope of the invention as defined by the appended claims. Further, in the detailed description of the exemplary embodiments, numerous specific details are set forth in order to provide a comprehensive understanding of the claimed invention. However, one skilled in the art would understand that various embodiments may be practiced without such specific details.

Although the features and elements of the present exemplary embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A method for determining a position of a portion of a subcutaneous medical device within a patient, the method comprising:
inserting the subcutaneous medical device into the patient, wherein a portion of the subcutaneous medical device includes a magnetic element, the magnetic element including at least two poles;
moving a magnetic detector, which includes a single magnetic sensor and is arranged outside of the patient, along the patient until static magnetic flux of the magnetic element is detected; and
determining, using a processor coupled to the single magnetic sensor, the position of the portion of the subcutaneous medical device within the patient based on a measurement of the detected static magnetic flux of the magnetic element by the single magnetic sensor without applying an external magnetic field to the magnetic element, the method further comprising
measuring the static magnetic flux of the magnetic element over a period of time while the magnetic sensor and magnetic element are in a fixed position relative to each other; and
determining whether or not the subcutaneous medical device is inserted into an artery in the patient based on changes in magnetic flux of the magnetic element over the period of time.

2. A method for determining a three-dimensional position of a portion of a subcutaneous medical device within a patient, the method comprising:
inserting the subcutaneous medical device into the patient, wherein the subcutaneous medical device includes a magnetic element, the magnetic element including at least two poles;
obtaining a static magnetic flux measurement from each of magnetic sensor of a magnetic sensor array; and
determining, using a processor coupled to the magnetic sensor array, the three-dimensional position, inclination angle, and orientation of the subcutaneous medical device within the patient based on a single static magnetic flux measurement from only two magnetic sensors of the magnetic sensor array without applying an external magnetic field to the magnetic element, the method further comprising
measuring the static magnetic flux of the magnetic element layover a period of time while the array of magnetic sensors and the magnetic element are in a fixed position relative to each other; and
determining whether or not the subcutaneous medical device is inserted into an artery in the patient based on changes in magnetic flux of the magnetic element layover the period of time.
